SEQUENCE LISTING (13 sequences)

SEQ ID NO: 1 - protein, 42 aa, Homo sapiens [sequence omitted]

SEQ ID NO: 2 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 126 aa, Homo sapiens [sequence omitted]

SEQ ID NO: 4 - protein, 110 aa, Homo sapiens
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 5 - protein, 10 aa, Homo sapiens
GFTFSSYAMS

SEQ ID NO: 6 - protein, 17 aa, Homo sapiens
AINASGTRTYYADSVKG

SEQ ID NO: 7 - protein, 17 aa, Homo sapiens
GKGNTHKPYGYVRYFDV

SEQ ID NO: 8 - protein, 12 aa, Homo sapiens
RASQSVSSSYLA

SEQ ID NO: 9 - protein, 7 aa, Homo sapiens
GASSRAT

SEQ ID NO: 10 - protein, 8 aa, Homo sapiens
LQIYNMPI

SEQ ID NO: 11 - protein, 940 aa, Artificial Sequence
  Heavy chain antibody construct
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 1168 aa, Artificial Sequence
  Heavy chain antibody construct
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 13 - protein, 215 aa, Artificial Sequence
  Light chain antibody construct
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0168253 A1
(43) Pub. Date: Jun. 16, 2016

(54) THERAPEUTIC FUSION PROTEIN

(71) Applicant: HOFFMANN-LA ROCHE INC., Nutley, NJ (US)

(72) Inventors: Bernd Bohrmann, Riehen (CH); Per-Ola Freskgard, Reinach BL (CH); Hendrik Knoetgen, Penzberg (DE); Jens Niewoehner, Muenchen (DE)

(73) Assignee: Hoffmann- La Roche Inc., Nutley, NJ (US)

(21) Appl. No.: 14/907,173

(22) PCT Filed: Jul. 30, 2014
(86) PCT No.: PCT/EP2014/066355

§ 371 Date: Jan. 22, 2016

(30) Foreign Application Priority Data
Aug. 2, 2013 (EP) 13179056.0

(51) Int. Cl. C07K 16/28; C12N 9/64; C07K 16/18

(52) U.S. Cl. CPC C07K 16/2881; C07K 16/18; C12N 9/6494; C07K 2317/55; C07K 2317/21; C07K 2319/50; C07K 2319/74; C07K 2317/565; C07K 2317/526; A61K 2039/505

(57) ABSTRACT

The invention relates to a fusion protein comprising an antibody directed to Aβ, a monovalent binding entity which binds to a blood brain barrier receptor and a neprilysin.

The present invention relates to a fusion protein comprising an antibody directed to Aβ, a monovalent binding entity which binds to a blood brain barrier receptor and a neprilysin moiety.

### BACKGROUND

About 70% of all cases of dementia are due to Alzheimer's disease which is associated with selective damage of brain regions and neural circuits critical for cognition. Alzheimer's disease is characterized by neurofibrillary tangles in particular in pyramidal neurons of the hippocampus and numerous amyloid plaques containing mostly a dense core of amyloid deposits and defused halos.

The extracellular neuritic plaques contain large amounts of a pre-dominantly fibrillar peptide termed “amyloid β”, “A-beta”, “Aβ4”, “β-A4” or “Aβ”; see Selkoe (1994), Ann. Rev. Cell Bio. 10, 373-403, Koo (1999), PNAS Vol. 96, pp. 9989-9990, U.S. Pat. No. 4,666,829 or Glenner (1984), BBRC 12, 1131. This amyloid is derived from “Alzheimer precursor protein/P-amyloid precursor protein” (APP). APPs are integral membrane glycoproteins (see Sisodia (1992), PNAS Vol. 89, pp. 6075) and are endoproteolytically cleaved within the Aβ sequence by a plasma membrane protease, α-secretase (see Sisodia (1992), Joe. cit.). Furthermore, further secretase activity, in particular β-secretase and γ-secretase activity leads to the extracellular release of amyloid-β (Aβ) comprising either 39 amino acids (Aβ39), 40 amino acids (Aβ40), 42 amino acids (Aβ42) or 43 amino acids (Aβ43); see Sinha (1999), PNAS 96, 11094-1053; Price (1998), Science 282, 1078 to 1083; WO 00/72880 or Hardy (1997), TINS 20, 154.

It is of note that Aβ has several naturally occurring forms, whereby the human forms are referred to as the above mentioned Aβ39, Aβ40, Aβ41, Aβ42 and Aβ43. The most prominent form, Aβ42, has the amino acid sequence (starting from the N-terminus):

DAEFRHDSGYEVHHQKLVFFAEDVGSNKGAIIGLMVGGVVIA (Seq. Id. No. 1). In Aβ41, Aβ40, Aβ39, the C-terminal amino acids A, IA and VIA are missing, respectively. In the Aβ43-form an additional threonine residue is comprised at the C-terminus of the above depicted sequence (Seq. Id. No. 1).

The time required to nucleate Aβ40 fibrils was shown to be significantly longer than that to nucleate Aβ42 fibrils; see P. T. Lansbury, Jr. and J. D. Harper (1997), Ann. Rev. Biochem. 66, 385-407. As reviewed in Wagner (1999), J. Clin. Invest. 104, 1239-1332, the Aβ42 is more frequently found associated with neuritic plaques and is considered to be more fibrillogenic in vitro. It was also suggested that Aβ42 serves as a “seed” in the nucleation-dependent polymerization of ordered non-crystalline Aβ peptides; Jarrett (1993), Cell 93, 1055-1058. Modified APP processing and/or the generation of extracellular plaques containing proteinaceous depositions are not only known from Alzheimer's pathology but also from subjects suffering from other neurological and/or neurodegenerative disorders. These disorders comprise, inter alia, Down's syndrome, Hereditary cerebral hemorrhage with amyloidosis Dutch type, Parkinson's disease, ALS (amyotrophic lateral sclerosis), Creutzfeld Jacob disease, HIV-related dementia and motor neuropathy.

Until now, only limited medical intervention schemes for amyloid-related diseases have been described. For example, cholinesterase inhibitors like galantamine, rivastigmine or donepezil have been discussed as being beneficial in Alzheimer's patients with only mild to moderate disease. However, also adverse events have been reported due to cholinergic action of these drugs. While these cholinergic-enhancing treatments do produce some symptomatic benefit, therapeutic response is not satisfactory for the majority of patients treated. It has been estimated that significant cognitive improvement occurs in only about 5% of treated patients and there is little evidence that treatment significantly alters the course of this progressive disease.

Consequently, there remains a tremendous clinical need for more effective treatments and in particular those which may arrest or delay progression of the disease. Also NMDA-receptor antagonists, like memantine, have been employed more recently.

However, adverse events have been reported due to the pharmacological activity. Further, such a treatment with these NMDA-receptor antagonists can merely be considered as a symptomatic approach and not a disease-modifying one.

Also immunomodulation approaches for the treatment of amyloid-related disorders have been proposed. WO 99/27944 discloses conjugates that comprise parts of the Aβ peptide and carrier molecules whereby said carrier molecule should enhance an immune response. Another active immunization approach is mentioned in WO 00172880, wherein also Aβ fragments are employed to induce an immune response.

Also passive immunization approaches with general anti-Aβ antibodies have been proposed in WO 99/27944 or WO 01/62801 and specific humanized antibodies directed against portions of Aβ have been described in WO 02/46237, WO 02/088306 and WO 02/088307. WO 00177178 describes antibodies binding a transition state adopted by β-amyloid during hydrolysis. WO 03/070760 discloses antibody molecules that recognize two discontinuous amino acid sequences on the Aβ peptide.

The technical problem underlying the present invention is to provide efficacious means and methods in the medical management of amyloid disorders, in particular means and methods for the reduction of detrimental amyloid plaques in patients in need of a medical intervention.

### SHORT DESCRIPTION OF THE FIGURES

FIG. 1 shows binding of mAb31 and trifunctional polypeptide TriGant to Aβ1-40 fibers in vitro by ELISA. Control antibody is a mouse transferrin receptor antibody (mouse TfR).

FIG. 2 shows binding binding of mAb31 and trifunctional polypeptide TriGant to murine transferrin receptor in vitro by FACS analysis.

FIG. 3 shows enzymatic activity of Neprilysin (R&D Systems, Cat. No 1182-ZNC) and trifunctional polypeptide TriGant in vitro.

FIG. 4 shows immunohistochemical staining of mAb31 and trifunctional polypeptide TriGant bound to native human β-amyloid plaques from brain sections of an Alzheimer's Disease patient.

FIG. 5 shows in vivo β-amyloid plaque decoration by mAb31 and trifunctional polypeptide TriGant in a mouse model of Alzheimer's disease. GAH555=goat anti-human IgG (H+L) conjugated to Alexa555 dye (Molecular Probes). BAP-2=a mouse monoclonal antibody against Aβ conjugated to Alexa 488.

### DETAILED DESCRIPTION OF EMBODIMENTS OF THE INVENTION

In a first aspect, the invention provides a fusion protein comprising an antibody directed to Aβ, a monovalent binding entity which binds to a blood brain barrier receptor and a neprilysin moiety.

In a particular embodiment of the invention the blood brain receptor is selected from the group consisting of the transferrin receptor, insulin receptor, insulin-like growth factor receptor, low density lipoprotein receptor-related protein 8, low density lipoprotein receptor-related protein 1 and heparin-binding epidermal growth factor-like growth factor.

In a particular embodiment of the invention, the monovalent binding entity of the fusion protein is a blood brain barrier ligand or a monovalent antibody fragment, preferably selected from scFv, Fv, scFab, Fab, VHH.

In a particular embodiment of the invention, the fusion protein comprises:
the antibody directed to Aβ comprising two heavy and two light chains,
the monovalent binding entity which binds to a blood brain barrier receptor is coupled to a C-terminal part of the first heavy chain of the antibody directed against Aβ by a first linker and
the neprilysin moiety is coupled to a C-terminal part of the second heavy chain of the antibody directed against Aβ by a second linker.

In a particular embodiment of the invention, the fusion protein comprises:
the antibody directed to Aβ comprising two heavy and two light chains,
the monovalent binding entity which binds to a blood brain barrier receptor is coupled to a C-terminal end of the Fc part of the first heavy chain of the antibody directed against Aβ by a first linker and
the neprilysin moiety is coupled to the C-terminal end of the Fc part of the second heavy chain of the antibody directed against Aβ by a second linker.

In a particular embodiment of the invention the first and second linker of the fusion protein are a peptide or a chemical linker.

In a particular embodiment of the invention, the monovalent binding entity of the fusion protein is a scFab directed to the transferrin receptor.

In a particular embodiment of the invention, the antibody of the fusion protein directed to Aβ comprises (a) H-CDR1 comprising the amino acid sequence of Seq. Id. No. 5, (b) H-CDR2 comprising the amino acid sequence of Seq. Id. No. 6, (c) H-CDR3 comprising the amino acid sequence of Seq. Id. No. 7, (d) L-CDR1 comprising the amino acid sequence of Seq. Id. No. 8, (e) L-CDR2 comprising the amino acid sequence of Seq. Id. No. 9 and (f) L-CDR3 comprising the amino acid sequence of Seq. Id. No. 10.

In a particular embodiment of the invention, the antibody of the fusion protein directed to Aβ comprises a V_{H} domain comprising the amino acid sequence of Seq. Id. No. 3 and a V_{L} domain comprising the amino acid sequence of Seq. Id. No. 4.

In a particular embodiment of the invention, the first heavy chain of the antibody of the fusion protein directed to Aβ comprises a first dimerization module and the second heavy chain of the antibody of the fusion protein directed to Aβ comprises a second dimerization module allowing heterodimerization of the two heavy chains.

In a particular embodiment of the invention, the first dimerization module of the first heavy chain of the antibody of the fusion protein directed to Aβ comprises knobs and the second dimerization module of the second heavy chain of the antibody of the fusion protein directed to Aβ comprises holes according to the knobs into holes strategy.

In a particular embodiment of the invention, the fusion protein is characterized by the presence of one single unit of the monovalent binding entity which binds to a blood brain barrier receptor, preferably the fusion protein is characterized by the presence of one single scFab directed to the transferrin receptor.

In a particular embodiment of the invention, the fusion protein comprises:
the antibody directed to Aβ comprising two heavy and two light chains,
a single unit of the monovalent binding entity which binds to a blood brain barrier receptor coupled to a C-terminal end of the Fc part of the first heavy chain of the antibody directed against Aβ by a first linker, preferably a single unit of a scFab directed to the transferrin recptor, and
a single unit of the neprilysin moiety coupled to the C-terminal end of the Fc part of the second heavy chain of the antibody directed against Aβ by a second linker.

In a particular embodiment of the invention, the neprilysin moiety derives from human neprilysin (Seq. Id. No. 2), more particularly the neprilysin moiety comprises amino acids 52-750 of human neprilysin i.e. amino acids 52-750 of Seq. Id. No. 2.

In a second aspect, the present invention relates to an isolated nucleic acid encoding the fusion protein of the present invention.

In a third aspect, the present invention relates to a host cell comprising the isolated nucleic acid of the present invention.

In a fourth aspect, the present invention relates to a pharmaceutical formulation comprising the fusion protein of the present invention and a pharmaceutical carrier.

The fusion proteins of the invention can be used as medicaments, in particular for the treatment of amyloid disorders, in particular for the treatment of Alzheimer's disease.

### DEFINITIONS

The knobs into holes dimerization modules and their use in antibody engineering are described in Carter P.; Ridgway J. B. B.; Presta L. G.: Immunotechnology, Volume 2, Number 1, February 1996, pp. 73-73(1)).

The “blood-brain barrier” or “BBB” refers to the physiological barrier between the peripheral circulation and the brain and spinal cord which is formed by tight junctions within the brain capillary endothelial plasma membranes, creating a tight barrier that restricts the transport of molecules into the brain, even very small molecules such as urea (60 Daltons). The BBB within the brain, the blood-spinal cord barrier within the spinal cord, and the blood-retinal barrier within the retina are contiguous capillary barriers within the CNS, and are herein collectively referred to an the blood-brain barrier or BBB. The BBB also encompasses the blood-CSF barrier (choroid plexus) where the barrier is comprised of ependymal cells rather than capillary endothelial cells.

The term “an antibody directed to Aβ” refers to an antibody that is capable of binding Aβ peptide with sufficient affinity such that the antibody is useful as a diagnostic and/or therapeutic agent in targeting Aβ peptide.

It is of note that Aβ has several naturally occurring forms, whereby the human forms are referred to as the above mentioned Aβ39, Aβ40, Aβ41, Aβ42 and Aβ43. The most prominent form, Aβ42, has the amino acid sequence (starting from the N-terminus):

DAEFRHDSGYEVHHQKLVFFAEDVGSNKGAIIGLMVGGVVIA (Seq. Id. No. 1). In Aβ41, Aβ40, Aβ39, the C-terminal amino acids A, IA and VIA are missing, respectively. In the Aβ43 form an additional threonine residue is comprised at the C-terminus of the above depicted sequence (Seq. Id. No. 1).

The “central nervous system” or “CNS” refers to the complex of nerve tissues that control bodily function, and includes the brain and spinal cord.

A “receptor at the blood-brain barrier” (abbreviated “R/BBB” herein) is an extracellular membrane-linked receptor protein expressed on brain endothelial cells which is capable of transporting molecules across the BBB or be used to transport exogenous administrated molecules. Examples of R/BBB herein include: transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is transferrin receptor (TfR).

The “effector entity” refers to a molecule that is to be transported to the brain across the BBB. The effector entity typically has a characteristic therapeutic activity that is desired to be delivered to the brain. Effector entities include neurologically disorder drugs and cytotoxic agents such as e.g. peptides, proteins and antibodies, in particular monoclonal antibodies.

The “monovalent binding entity” refers to a molecule able to bind specifically and in a monovalent binding mode to an R/BBB. The monovalent binding entity can for example be a part of an IgG such as a single scFab fragment. The monovalent binding entity can for example be a scaffold protein engineered using state of the art technologies like phage display or immunization. The monovalent binding entity can also be a peptide.

The “monovalent binding mode” refers to a specific binding to the R/BBB where the interaction between the monovalent binding entity and the R/BBB take place through one single epitope. The monovalent binding mode prevents any dimerization/multimerization of the R/BBB due to a single epitope interaction point. The monovalent binding mode prevents that the intracellular sorting of the R/BBB is changed.

The “transferrin receptor” (“TfR”) is a transmembrane glycoprotein (with a molecular weight of about 180,000) composed of two disulphide-bonded sub-units (each of apparent molecular weight of about 90,000) involved in iron uptake in vertebrates. In one embodiment, the TfR herein is human TfR comprising the amino acid sequence as in Schneider et al. Nature 311: 675-678 (1984), for example.

The term “antibody” herein is used in the broadest sense and specifically covers monoclonal antibodies, polyclonal antibodies, multispecific antibodies {e.g. bispecific antibodies) formed from at least two intact antibodies, and antibody fragments so long as they exhibit the desired biological activity.

“Antibody fragments” herein comprise a portion of an intact antibody which retains the ability to bind antigen. Examples of antibody fragments include Fab, Fab′, F(ab′)₂, and Fv fragments; diabodies; linear antibodies; single-chain antibody molecules such as scFv and scFab; and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. Specific examples of monoclonal antibodies herein include chimeric antibodies, humanized antibodies, and human antibodies, including antigen-binding fragments thereof. The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al, Proc. Natl. Acad. Sci. USA, 81:6851-6855 (1984)). Chimeric antibodies of interest herein include “primatized” antibodies comprising variable domain antigen-binding sequences derived from a non-human primate {e.g. Old World Monkey, such as baboon, rhesus or cynomolgus monkey) and human constant region sequences (U.S. Pat. No. 5,693,780).

“Humanized” forms of non-human {e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al, Nature 321:522-525 (1986); Riechmann et al, Nature 332:323-329 (1988); and Presta, Curr. Op. Struct. Biol 2:593-596 (1992).

A “human antibody” herein is one comprising an amino acid sequence structure that corresponds with the amino acid sequence structure of an antibody obtainable from a human B-cell, and includes antigen-binding fragments of human antibodies. Such antibodies can be identified or made by a variety of techniques, including, but not limited to: production by transgenic animals {e.g., mice) that are capable, upon immunization, of producing human antibodies in the absence of endogenous immunoglobulin production (see, e.g., Jakobovits et al, Proc. Natl Acad. Sci. USA, 90:2551 (1993); Jakobovits et al, Nature, 362:255-258 (1993); Bruggermann et al, Year in Immuno., 7:33 (1993); and U.S. Pat. Nos. 5,591,669, 5,589,369 and 5,545,807)); selection from phage display libraries expressing human antibodies or human antibody fragments (see, for example, McCafferty et al, Nature 348:552-553 (1990); Johnson et al, Current Opinion in Structural Biology 3:564-571 (1993); Clackson et al, Nature, 352:624-628 (1991); Marks et al, J. Mol. Biol. 222:581-597 (1991); Griffith et al, EMBO J. 12:725-734 (1993); U.S. Pat. Nos. 5,565,332 and 5,573,905); generation via in vitro activated B cells (see U.S. Pat. Nos. 5,567,610 and 5,229,275); and isolation from human antibody producing hybridomas.

A “multispecific antibody” herein is an antibody having binding specificities for at least two different epitopes. Exemplary multispecific antibodies may bind both an R/BBB and a brain antigen. Multispecific antibodies can be prepared as full-length antibodies or antibody fragments (e.g. F(ab′)₂ bispecific antibodies). Engineered antibodies with two, three or more (e.g. four) functional antigen binding sites are also contemplated (see, e.g., US Appln No. US 2002/0004587 A1, Miller et al.). Multispecific antibodies can be prepared as full length antibodies or antibody fragments.

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

The “variable domain” (variable domain of a light chain (V_{L}), variable domain of a heavy chain (V_{H})) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The antibody herein may be a “glycosylation variant” such that any carbohydrate attached to the Fc region, if present, is altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) describing antibodies with modified glycosylation. The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof.

Antibody “effector functions” refer to those biological activities attributable to the Fc region (a native sequence Fc region or amino acid sequence variant Fc region) of an antibody. Examples of antibody effector functions include Clq binding, complement dependent cytotoxicity (CDC), Fc receptor binding, antibody-dependent cell-mediated cytotoxicity (ADCC), etc. In one embodiment, the antibody herein essentially lacks effector function.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known. The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia, coli cells or Bacillus subtilis cells, etc.

As used herein, “specifically binding” or “binds specifically to” refers to an antibody selectively or preferentially binding to an antigen. The binding affinity is generally determined using a standard assay, such as ELISA or surface plasmon resonance technique (e.g. using BIACORE®).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “linker” as used herein is a structure that covalently or non-covalently connects the effector entity to the monovalent binding entity. In certain embodiments, a linker is a peptide. In other embodiments, a linker is a chemical linker.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al, J. Chromatogr. B 848:79-87 (2007).

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

In some embodiments, antibodies of the invention are used to delay development of a disease or to slow the progression of a disease.

Pharmaceutical Formulations

Therapeutic formulations of the fusion protein used in accordance with the present invention are prepared for storage by mixing with optional pharmaceutically acceptable carriers, excipients or stabilizers {Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary, optionally those with complementary activities that do not adversely affect each other. The type and effective amounts of such medicaments depend, for example, on the amount of fusion protein present in the formulation, and clinical parameters of the subjects. Exemplary such medicaments are discussed below.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes. In one embodiment the formulation is isotonic.

In one aspect, the fusion protein of the invention for use as a medicament is provided. In further aspects, the fusion protein of the invention for use in treating a neurological disease or disorder is provided such as amyloid disorders, in particular Alzheimer's disease. In certain embodiments, the fusion protein of the invention for use in a method of treatment is provided. In certain embodiments, the invention provides the fusion protein of the invention for use in a method of treating an individual having a neurological disease or disorder comprising administering to the individual an effective amount of the fusion protein of the invention. An “individual” according to any of the above embodiments is optionally a human.

The fusion protein of the invention can be used either alone or in combination with other agents in a therapy. For instance, the fusion protein of the invention may be co-administered with at least one additional therapeutic agent. In certain embodiments, an additional therapeutic agent is a therapeutic agent effective to treat the same or a different neurological disorder as the fusion protein of the invention is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug.

Such combination therapies noted above encompass combined administration (where two or more therapeutic agents are included in the same or separate formulations), and separate administration, in which case, administration of the fusion construct of the invention can occur prior to, simultaneously, and/or following, administration of the additional therapeutic agent and/or adjuvant. Fusion proteins of the invention can also be used in combination with other interventional therapies such as, but not limited to, radiation therapy, behavioral therapy, or other therapies known in the art and appropriate for the neurological disorder to be treated or prevented. The monovalent binding entity against an R/BBB of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration.

Dosing can be by any suitable route, e.g. by injections, such as intravenous or subcutaneous injections, depending in part on whether the administration is brief or chronic. Various dosing schedules including but not limited to monovalent or multiple administrations over various time-points, bolus administration, and pulse infusion are contemplated herein.

Lipid-based methods of transporting the fusion construct or a compound across the BBB include, but are not limited to, encapsulating the fusion construct or a compound in liposomes that are coupled to monovalent binding entity that bind to receptors on the vascular endothelium of the BBB (see e.g., U.S. Patent Application Publication No. 20020025313), and coating the monovalent binding entity in low-density lipoprotein particles (see e.g., U.S. Patent Application Publication No. 20040204354) or apolipoprotein E (see e.g., U.S. Patent Application Publication No. 20040131692).

For the prevention or treatment of disease, the appropriate dosage of fusion protein of the invention (when used alone or in combination with one or more other additional therapeutic agents) will depend on the type of disease to be treated, the type of fusion construct, the severity and course of the disease, whether the antibody is administered for preventive or therapeutic purposes, previous therapy, the patient's clinical history and response to the fusion construct, and the discretion of the attending physician. The fusion protein is suitably administered to the patient at one time or over a series of treatments. Depending on the type and severity of the disease, about 1 μg/kg to 15 mg/kg (e.g. 0.1 mg/kg-10 mg/kg) of fusion construct can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

### EXAMPLES

### Fusion Polypeptides Used in the Examples

The trifunctional polypeptide (TriGant) further characterized in the examples comprises a full length antibody directed to Abeta (MAB31), a single Fab directed to the transferrin receptor and neprilysin.

Anti Abeta antibody: Mab 31=Gantenerumab (INN). Single Fab (scFab) to transferrin receptor: mouse 8D3 anti-transferrin antibody (Boado, R. J. Zhang, Y. Wang, Y and Pardridge, W. M., Biotechnology and Bioengineering (2009) 102, 1251-1258).

The sequences of the heavy chains and the variable chain of the trifunctional polypeptides of the examples are as follows:

Mab 31 heavy chain Knob—sFab 8D3: MAB31-IgG1-KNOB-SS_G4S-4_VL-8D3-CK_G4S-6-GG_VH-8D3-CH1 (Seq. Id. No. 11)

Composition of Seq. Id. No. 11:
Mab31 human IgG1 heavy chain without C-terminal Lys
Glycine Serine-linker
Variable light chain domain (VL) variant (L596V and L5981) of the mouse 8D3 anti-transferrin antibody (Boado, R. J. Zhang, Y. Wang, Y and Pardridge, W. M., Biotechnology and Bioengineering (2009) 102, 1251-1258)
Human C-kappa light chain
Glycine Serine-linker
Variable heavy chain domain (VH) of the mouse 8D3 anti-transferrin antibody

(Boado, R. J. Zhang, Y. Wang, Y and Pardridge, W. M., Biotechnology and Bioengineering (2009) 102, 1251-1258)
Human IgG1 CH3 heavy chain domain

Mab 31 heavy chain Hole—Neprilysin: MAB31_8 D3_HC-HOLE_NEPRI (Seq. Id. No. 12).

Composition of Seq. Id. No. 12:
Mab31 human IgG1 heavy chain without C-terminal Lys
Glycine Serine-linker
Amino acids 52-750 of Seq. Id. No. 2 (human Neprilysin) Mab 31 light chain: TPIP:5170_MAB31-LC (Seq. Id. No. 13).

### Example 1

### Determination of Binding to Aβ1-40 Fibers In Vitro by ELISA

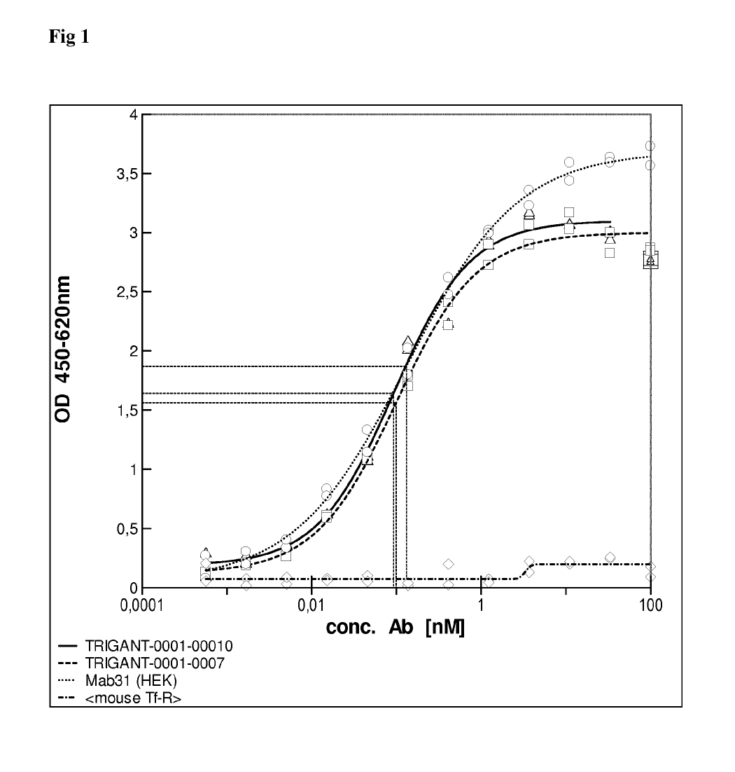

Binding of fusion polypeptide to fibrillar Aβ was measured by an ELISA assay. Briefly, A13(1-40) was coated at 7 μg/mL in PBS onto Maxisorp plates for 3 days at 37° C. to produce fibrillar Abeta, then dried for 3 h at RT. The plate was blocked with 1% Crotein C and 0.1% RSA in PBS (blocking buffer) for 1 h at RT, then washed once with wash buffer. Fusion polypeptides or controls were added at concentrations up to 100 nM in blocking buffer and incubated at 4° C. overnight. After 4 wash steps, constructs were detected by addition of anti-human-IgG-HRP (Jackson Immunoresearch) at 1:10,000 dilution in blocking buffer (1 RT), followed by 6 washes and incubation in TMB (Sigma). Absorbance was read out at 450 nm after stopping color development with 1 N HCl (see FIG. 1).

### Example 2

### Determination of Binding to Murine Transferrin Receptor In Vitro

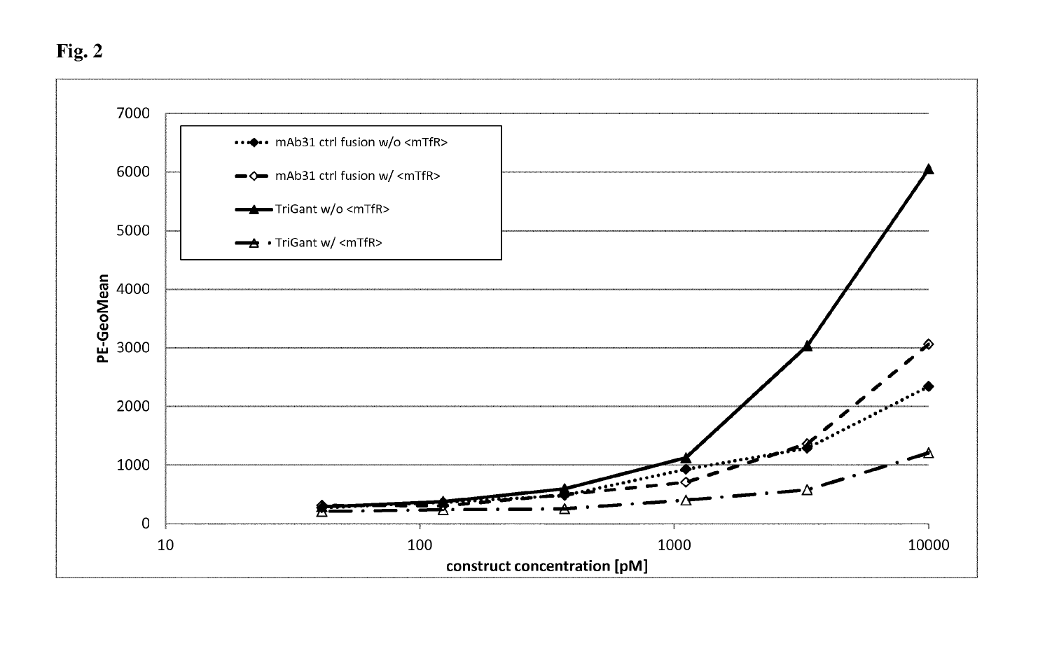

Binding of fusion polypeptide to murine transferrin receptor was tested by FACS analysis on mouse X63.AG8-563 myeloma cells. As Aβ antibody mAb31(HEK) showed a certain tendency to unspecifically bind to Ag8 cells, specific binding was quantified by co-incubation with a 20fold excess of anti-mouse-TfR antibody. Cells were harvested by centrifugation, washed once with PBS and 5×104 cells incubated with a 1.5 pM to 10 nM dilution series of the polypeptide fusions with or without addition of 200 nM anti-mouse TfR antibody in 100 μL RPMI/10% FCS for 1.5 h on ice. After 2 washes with RPMI/10% FCS, cells were incubated with goat-anti-human IgG coupled to Phycoerythrin (Jackson Immunoresearch) at a dilution of 1:600 in RPMI/19% FCS for 1.5 h on ice. Cells were again washed, resuspended in RPMI/10% FCS and Phycoerythrin fluorescence measured on a FACS-Array instrument (Becton-Dickinson) (see FIG. 2).

### Example 3

### Determination of Enzymatic Activity of Neprilysin In Vitro (Apparent Km)

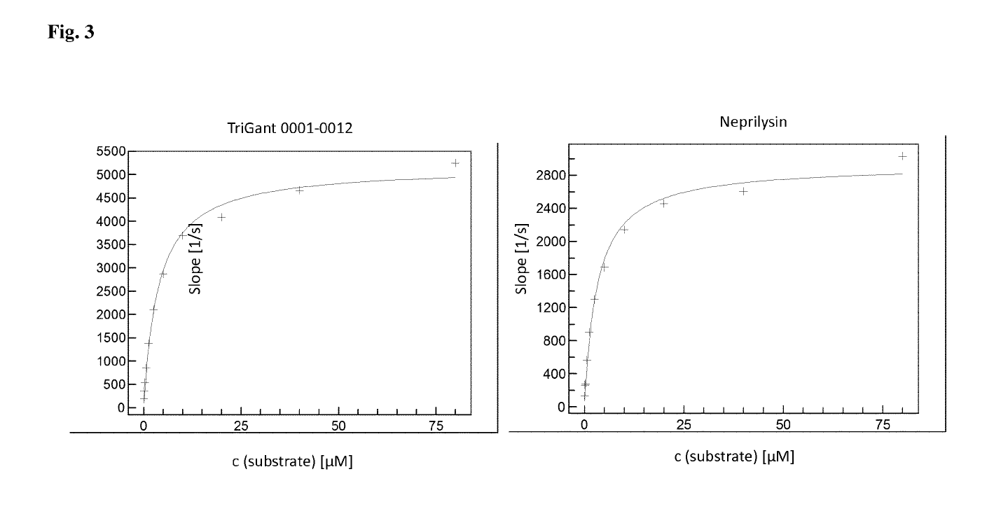

The 20 μl assay was performed on low-volume black Costar 384-well plates at 25° C. A working solution of 160 μM peptide substrate MCA-RPPGFSAFK(Dnp)-OH (R&D Systems Cat. No. ES005) was prepared in 50 mM Tris-HCl pH7.8, 25 mM NaCl and 5 mM ZnCl₂. 10 μl of Neprilysin (R&D Systems, Cat. No 1182-ZNC) or Neprilysin fusion polypeptide, diluted to 1 nM in assay buffer, were transferred to plate. For determination of apparent Km values various concentrations of substrate (0.078-80 nM in 2-fold dilutions dilutions) were added and the enzyme reaction started. The fluorescence increase was monitored with excitation at 320 nm and emission at 405 nm on an Envision Reader. Hydrolysis rates and apparent Km values were calculated using XLFit® software (IDBS) (see FIG. 3 and table 1)

TABLE 1

| | Vmax | Km |
|---|---|---|
| | [1/s] | [μM] |
| TriGant 0001-0012 | 5169 | 3.8 |
| Neprilysin (RnD) | 2926 | 3.2 |

### Example 4

### Staining of Fusion Polypeptide to Native Human β-Amyloid Plaques from Brainsections of an Alzheimer's Disease Patient by Indirect Immunofluorescence

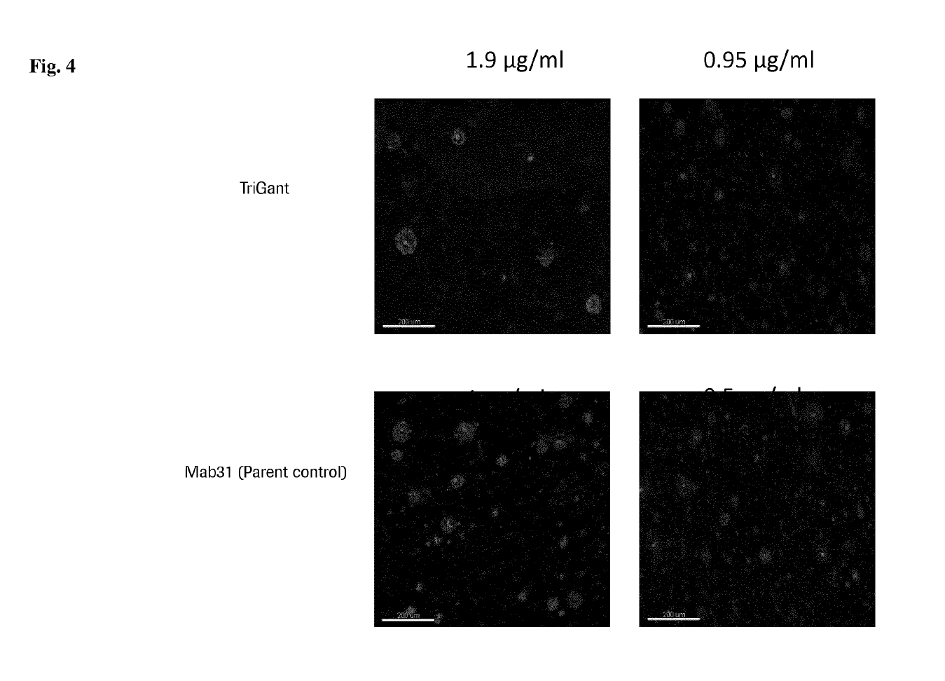

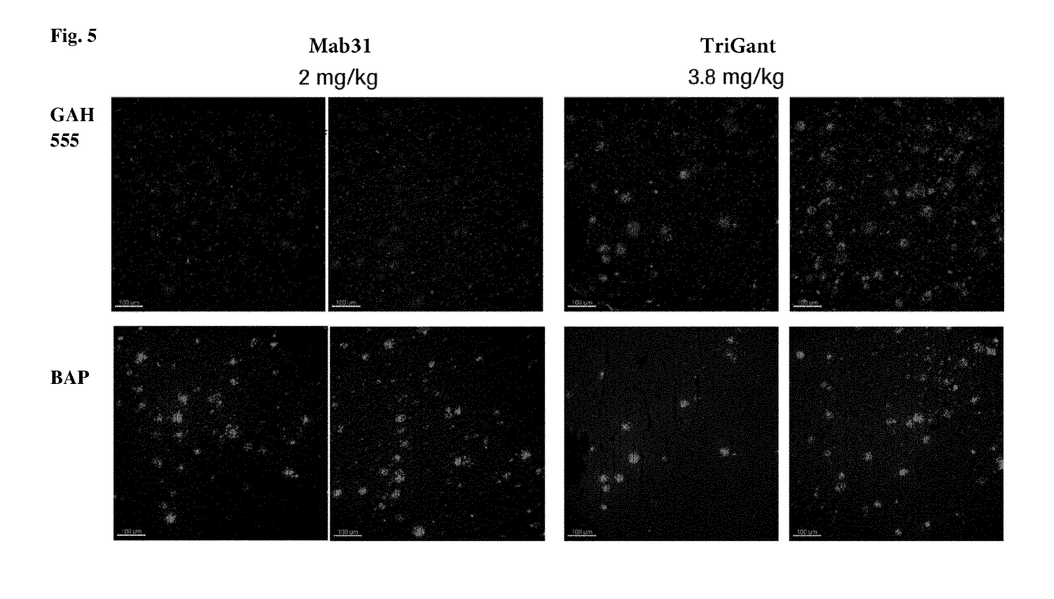

Fusion polypeptide was tested for the ability to stain native human β-amyloid plaques by immunohistochemistry analysis using indirect immunofluorescence. Specific and sensitive staining of genuine human β-amyloid plaques was demonstrated. Cryostat sections of unfixed tissue from the temporal cortex obtained postmortem from patients positively diagnosed for Alzheimer's disease were labeled by indirect immunofluorescence. A two-step incubation was used to detect bound fusion polypeptide, which was revealed by affinity-purified goat anti-human (GAH555) IgG (H+L) conjugated to Alexa 555 dye (Molecular Probes). Controls included unrelated human IgG1 antibodies (Sigma) and the secondary antibody alone, which all gave negative results. All types of β-amyloid plaques were clearly and consistently revealed at fusion polypeptide concentrations tested from 10 ng/ml to 5 μg/ml Specific and sensitive staining of genuine human amyloid-β plaques is shown for fusion polypeptide at a concentration of 0.95 μg/ml and 1.9 μg/ml (see FIG. 4).

### Example 5

### In Vivo β-Amyloid Plaque Decoration by Fusion Polypeptide in a Mouse Model of Alzheimer's Disease

Fusion polypeptide was tested in APP/PS2 double transgenic mice, a mouse model for AD-related amyloidosis (Richards (2003), J. Neuroscience, 23, 8989-9003) for their ability to immuno-decorate β-amyloid plaques in vivo. This enabled assessment of the extent of brain penetration and binding to amyloid-β plaques. The fusion polypeptide was administered at different doses compared to naked anti-Aβ monoclonal antibody and after 6 days animals were perfused with phosphate-buffered saline and the brains frozen on dry ice and prepared for cryosectioning. The fusion polypeptide showed substantially improved and highly effective brain penetration in vivo (as compared to the naked anti-Aβ monoclonal antibody).

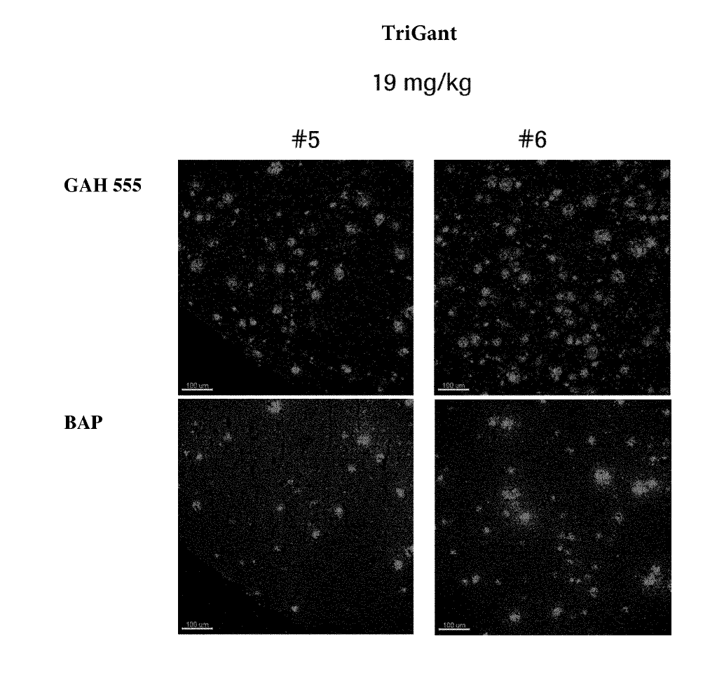

The presence of the antibodies bound to β-amyloid plaques was assessed using unfixed cryostat sections either by single-labeled indirect immunofluorescence with goat anti-human IgG (H+L) conjugated to Alexa555 dye (GAH555) (Molecular Probes) at a concentration of 15 μg/ml for 1 hour at room temperature. A counterstaining for amyloid plaques was done by incubation with BAP-2, a mouse monoclonal antibody against Aβ conjugated to Alexa 488 at a concentration of 0.5 μg/ml for 1 hour at room temperature. Slides were embedded with fluorescence mounting medium (S3023 Dako) and imaging was done by confocal laser microscopy (FIG. 5).

At equimolar doses (2 and 3.8 mg/kg) fusion polypeptide were found to cross substantially better the blood brain barrier and strongly immuno-decorate all β-amyloid plaques in vivo. Representative images shown in FIG. 5 demonstrate the improved binding capacity of the fusion polypeptide compared to the naked monoclonal antibody which crosses the blood-brain border at much lower extent.

### Example 6

### Recombinant Production of Fusion Polypeptide

DNA Preparation:

500 ml or 5 L of overnight bacterial LB culture were harvested and plasmid DNA was extracted according to the manufacturer's protocol (High speed Maxi kit, Qiagen, Cat. No. 12663). The resulting plasmid DNA was eluted in 1 ml TE buffer and DNA concentration was determined by spectrophotometric measurement (Epoch, BioTek).

Expression Plasmids:

Expression plasmids comprising expression cassettes for the expression of the heavy and light chains were separately assembled in mammalian cell expression vectors.

General information regarding the nucleotide sequences of human light and heavy chains from which the codon usage can be deduced is given in: Kabat, E. A., et al., Sequences of Proteins of Immunological Interest, 5th ed., Public Health Service, National Institutes of Health, Bethesda, Md. (1991), NIH Publication No 91-3242.

a) Antibody without Neprilysin:

The transcription unit of the κ-light chain is composed of the following elements:
the immediate early enhancer and promoter from the human cytomegalovirus (hCMV) including a 5′UTR of a human light chain germline gene,
the light chain variable region including the signal peptide sequence encoding genomic DNA segment (L1, signal-sequence-intron, L2) of a human germline gene,
the human κ-light gene constant region including the mouse κ-light gene intron 2, and
the SV40 polyadenylation (“poly A”) signal sequence.

The transcription unit of the γ1-heavy chain is composed of the following elements:
the immediate early enhancer and promoter from the human cytomegalovirus (HCMV) including the 5′-UTR of a human heavy chain germline gene,
the γ1-chain variable region including the signal peptide sequence encoding genomic DNA segment (L1, signal-sequence-intron, L2) of a human germline gene,
the genomic human γ1-heavy chain gene constant region including the mouse heavy chain intron 2;
the SV40 polyadenylation (“poly A”) signal sequence.

Further the plasmid contains
a neomycin resistance gene,
an origin of replication from the vector pUC18 which allows replication of this plasmid in E. coli, and
a β-lactamase gene which confers ampicillin resistance in E. coli.

b) Antibody with Neprilysin Fused to the C-Terminus of the Heavy Chain:

The expression plasmid for the light chain comprises
the transcription unit of the κ-light chain composed of the following elements:
the immediate early enhancer and promoter from the human cytomegalovirus (hCMV) including a 5′UTR of a human light chain germline gene,
the light chain variable region including the signal peptide sequence encoding genomic DNA segment (L1, signal-sequence-intron, L2) of a murine germline gene,
the human κ-light gene constant region including the mouse κ-light gene intron 2, and
the BGH polyadenylation (“poly A”) signal sequence;
a neomycin resistance gene,
an origin of replication from the vector pUC18 which allows replication of this plasmid in E. coli, and
a β-lactamase gene which confers ampicillin resistance in E. coli.

The expression plasmid for the heavy chain comprises
the transcription unit of the γ1-heavy chain composed of the following elements:
the immediate early enhancer and promoter from the human cytomegalovirus (HCMV) including the 5′-UTR of a human heavy chain germline gene,
the γ1-chain variable region including the signal peptide sequence encoding genomic DNA segment (L1, signal-sequence-intron, L2) of a human germline gene,
the genomic human γ1-heavy chain gene constant region including the mouse heavy chain intron 2;
a neprilysin encoding nucleic acid,
the BGH polyadenylation (“poly A”) signal sequence;
an origin of replication from the vector pUC18 which allows replication of this plasmid in E. coli, and
a β-lactamase gene which confers ampicillin resistance in E. coli.

Transfection:

HEK293 cells were diluted to 8×105 cells/ml the day before transfection. About 1 to 1.6×106 cells/ml were transfected according to the manufacturer's protocol. For a final transfection volume of 1000 ml, 1000 μg DNA were diluted to a final volume of 50 ml with Opti-MEM® I Reduced Serum Medium (Gibco, Cat. No. 31985070). Two microliter of 293fectinTMReagent (Invitrogen, Cat. No. 12347019) per 1 μg DNA were equally diluted to a final volume of 1 ml with Opti-MEM® medium and incubated for 5 minutes. After incubation the diluted DNA was added to the diluted 293fectinTMReagent, gently mixed, incubated for another 20-30 minutes and afterwards dropwise pipetted to 950 ml of the HEK293 cell suspension to obtain a final volume of 1000 ml. The cells were incubated under cell culture condition (37° C., 8% CO2, 80% humidity) on an orbital shaker rotating at 125 rpm and harvested after 72 hours. The harvest was centrifuged for 10 minutes at 1000 rpm followed by 10 minutes at 3000 rpm and filtered through a 22 μm sterile filter (Millipore, Cat. No. SCGPUO5RE).

Purification:

Cells were removed from culture medium by centrifugation. Complexes were purified from supernatants by Protein A affinity chromatography (MabSelect-Sepharose on a ÄKTA-Avant). Eluted complexes were concentrated with Amicon centrifugation tubes to a protein concentration of 3 mg/ml. An aliquot was analyzed on a size exclusion chromatography (HPLC TSKgel GFC300 Sys89). Preparative SEC on a Superdex 200 was performed to remove aggregates and to buffer the fusion proteins in 20 mM histidine, 140 mM NaCl, pH 6.0. Eluted complexes were concentrated with Amicon centrifugation tube to a protein concentration of 1 mg/ml and sterile filtered (0.2 μm pore size).

Analytics:

Complex samples were analyzed by OD280 using a UV spectrophotometer to determine the protein concentration in solution. The extinction coefficient required for this was calculated from the amino acid sequence according to Pace (Pace et al., Protein Science 4 (1995) 2411-2423). Size-exclusion chromatography (SE-HPLC) was performed on TSK-Ge1300SWXL or Superdex 200 columns with a 0.2 M potassium phosphate buffer, comprising 0.25 M KCl, pH 7.0 as mobile phase in order to determine the content of monomeric, aggregated and degraded species in the samples. Sodium dodecyl sulfate (SDS) polyacrylamide gel electrophoresis (reducing and non-reducing) was performed to analyze the purity of the complex preparations with regard to product-related degradation products and unrelated impurities. Electrospray ionization mass spectrometry (ESI-MS) was performed with reduced (TCEP) and deglycosylated (N-glycosidase F) samples to confirm the correct mass/identity of each chain and detect chemical modifications. ESI-MS of the deglycosylated samples was carried out to analyze the nature and quality of the fully assembled protein and detect potential product-related side products (table 2).

TABLE 2

| | | | protein after | | | total |
|---|---|---|---|---|---|---|
| | | cultivation | Protein A | monomer | aggregate | monomeric |
| | | volume | purification | content | content | protein |
| antibody | neprilysin | [l] | [mg/l] | [%] | [%] | [mg/l] |
| Mab 31 | no | 2.4 | 9.5 | >98% | <2% | 9.3 |
| Mab 31 | yes | 14 | 20.1 | 90.8 | 7.3 | 18.3 |

Results:

It can be seen that fusion of the neprilysin moiety to one of the antibody heavy chains of Mab31 increased the expression yield of the fusion polypeptide compared to the yield of the Mab31 antibody.

Polypeptide Sequences of the Invention

| | Sequence Identification |
|---|---|
| Polypeptide | Number (Seq. Id. No.) |
| Abeta peptide | 1 |
| Human Neprilysin | 2 |
| Mab 31 V_{H} | 3 |
| Mab 31 V_{L} | 4 |
| Mab 31 V_{H} CDR1 | 5 |
| Mab 31 V_{H} CDR2 | 6 |
| Mab 31 V_{H} CDR3 | 7 |
| Mab 31 V_{L} CDR1 | 8 |
| Mab 31 V_{L} CDR 2 | 9 |
| Mab 31 V_{L} CDR3 | 10 |
| Mab 31 heavy chain Knob - sFab 8D3 | 11 |
| Mab 31 heavy chain Hole - Neprilysin | 12 |
| Mab 31 light chain | 13 |

1. A fusion protein comprising, an antibody directed to Aβ, a monovalent binding entity which binds to a blood brain barrier receptor and a neprilysin moiety.

2. The fusion protein of claim 1, wherein the blood brain receptor is selected from the group consisting of the transferrin receptor, insulin receptor, insulin-like growth factor receptor, low density lipoprotein receptor-related protein 8, low density lipoprotein receptor-related protein 1 and heparin-binding epidermal growth factor-like growth factor.

3. The fusion protein of claim 1, wherein the monovalent binding entity is a blood brain barrier ligand or a monovalent antibody fragment.

4. The fusion protein of claim 1, comprising:
the antibody directed to Aβ comprising two heavy and two light chains,
the monovalent binding entity which binds to a blood brain receptor coupled to a C-terminal part of the first heavy chain of the antibody directed against Aβ by a first linker and
the neprilysin moiety coupled to a C-terminal part of the second heavy chain of the antibody directed against Aβ by a second linker.

5. The fusion protein of claim 4, wherein the monovalent binding entity which binds to a blood brain receptor is coupled to the C-terminal end of the Fc part of the first heavy chain of the full length antibody directed against Aβ by a first linker and
the neprilysin moiety is coupled to the C-terminal end of the Fc part of the second heavy chain of the full length antibody directed against Aβ by a second linker.

6. The fusion protein of claim 5, wherein the first and second linker are a peptide or a chemical linker.

7. The fusion protein of claim 1, wherein the monovalent binding entity which binds to a blood brain barrier receptor is a scFab directed to the transferrin receptor.

8. The fusion protein of claim 1, wherein the antibody directed to Aβ comprises (a) H-CDR1 comprising the amino acid sequence of Seq. Id. No. 5, (b) H-CDR2 comprising the amino acid sequence of Seq. Id. No. 6, (c) H-CDR3 comprising the amino acid sequence of Seq. Id. No. 7, (d) L-CDR1 comprising the amino acid sequence of Seq. Id. No. 8, (e) L-CDR2 comprising the amino acid sequence of Seq. Id. No. 9 and (f) L-CDR3 comprising the amino acid sequence of Seq. Id. No. 10.

9. The fusion protein of claim 1, wherein the antibody directed to Aβ comprises a V_{H} domain comprising the amino acid sequence of Seq. Id. No. 3 and a V_{L} domain comprising the amino acid sequence of Seq. Id. No. 4.

10. The fusion protein of claim 4, wherein one of the heavy chains of the antibody directed to Aβ comprises a first dimerization module and the second heavy chain of the antibody directed to Aβ comprises a second dimerization module allowing heterodimerization of the two heavy chains.

11. The fusion protein of claim 10, wherein the first dimerization module comprises knobs and the second dimerization module comprises holes according to the knobs into holes strategy.

12. The fusion protein of claim 1, comprising one monovalent binding entity which binds to a blood brain barrier receptor.

13. An isolated nucleic acid encoding the fusion protein of claim 1.

14. A host cell comprising the nucleic acid of claim 13.

15. A pharmaceutical formulation, comprising the fusion protein of claim 1 and a pharmaceutical carrier.

16-17. (canceled)

18. The fusion protein of claim 3, wherein the monovalent binding entity is a blood brain barrier ligand or a monovalent antibody fragment selected from the group consisting of scFv, Fv, scFab, Fab and VHH.

19. The fusion protein of claim 6, wherein the first and second linker are a peptide linker.

20. The fusion protein of claim 12, wherein the monovalent binding entity which binds to a blood brain barrier receptor is scFab directed to the transferrin receptor.
